SEQUENCE LISTING (4 sequences)

SEQ ID NO: 1 - DNA, 32 bp, Artificial sequence
  Primer 291012jvi4
gccgcaattgtggctgcaggtcgaccatgccg

SEQ ID NO: 2 - DNA, 33 bp, Artificial sequence
  Primer 291012jvi5
gccgcaattgaatgataccacagtctagttgac

SEQ ID NO: 3 - DNA, 32 bp, Artificial sequence
  Primer BGMH155
ggacttaanagcgagagagttgaacctggacg

SEQ ID NO: 4 - DNA, 35 bp, Artificial sequence
  Primer BGMH156
gggtttaancagatggcccgagaggactattccga

(19) United States
(12) Patent Application Publication
(10) Pub. No.: US 2016/0304905 A1
(43) Pub. Date: Oct. 20, 2016

(54) Fungal Gene Library By Double Split-Marker Integration

(71) Applicant: NOVOZYMES A/S, Bagsvaerd (DK)

(72) Inventors: Bjarne G. Hansen, Allerod (DK); Carsten Lillelund Olsen, Bagsvaerd (DK); Jesper Vind, Vaerlose (DK)

(21) Appl. No.: 15/101,252

(22) PCT Filed: Dec. 3, 2014
(86) PCT No.: PCT/EP2014/076395

§ 371 Date: Jun. 2, 2016

(30) Foreign Application Priority Data
Dec. 3, 2013 (EP) 13195574.2

(51) Int. Cl. C12N 15/90; C12N 15/80

(52) U.S. Cl. CPC C12N 15/902; C12N 15/80

(57) ABSTRACT

The present invention relates to a method for site-specific chromosomal integration of a gene library in a filamentous fungal host cell and a polynucleotide construct suitable for this purpose.

### REFERENCE TO SEQUENCE LISTING

This application contains a Sequence Listing in computer readable form. The computer readable form is incorporated herein by reference.

### FIELD OF THE INVENTION

The present invention relates to a combination of several known features and techniques which, when applied together as shown herein, provide an improved method for site-specific chromosomal integration of a gene library in a filamentous fungal host cell and a polynucleotide construct suitable for this purpose.

### BACKGROUND OF THE INVENTION

Autosomally replicating plasmids have been employed to successfully transform fungal host cells, both as pre-formed circular plasmids and as linearized plasmids co-transformed together with PCR fragments for the purpose of in vivo recombination within the transformed host cell. Fungal replicating plasmids are equipped with an autonomous replicating sequence, such as, the well-known AMA1 sequence originally isolated from Aspergillus nidulans or one of the known functional derivatives thereof (See, for example, Aleksenko and Clutterbuck, Mol Microbiol. 1996 February; 19(3):565-74).

Site-specific split-marker or bi-partite chromosomal integration in filamentous fungal hosts was disclosed, e.g. by Nielsen et al (Efficient PCR-based gene targeting with a recyclable marker for Aspergillus nidulans, Fungal Genetics and Biology 43 (2006) 54-64).

### SUMMARY OF THE INVENTION

It is well-known that the use of autosomally replicating plasmids improves transformation efficiency in filamentous fungal host cells, as well as provides consistent expression levels to enable comparisons of expressed proteins from a gene library comprised in said plasmids when introduced into a host cell. The inventors of the instant application, however, have found that transformation of a filamentous fungal host cell with an autonomously replicating vector followed by site-specific split-marker chromosomal integration of a lipase-encoding gene comprised in said vector provided a superior transformation efficiency combined with a surprising increase in expression level of the lipase as evaluated by SDS-PAGE (see FIG. 3).

Accordingly, in a first aspect, the invention relates to methods for site-specific chromosomal integration of a gene library in a filamentous fungal host cell, comprising the steps of:
a) providing a polynucleotide construct comprising an autonomous replication sequence and an integration cassette, said cassette comprising the gene library and a first selectable marker, wherein the cassette is flanked on one side by a non-functional part of a second selectable marker and on the other side by a non-functional part of a third selectable marker;
b) providing a filamentous fungal host cell comprising in its chromosome a non-functional part of the second selectable marker and a non-functional part of the third selectable marker, wherein correct recombinations between the chromosomal non-functional parts of the second and third selectable markers with the respective non-functional parts in the polynucleotide construct will result in functional chromosomal second and third selectable markers;
c) transforming the host cell with the polynucleotide construct and selecting for the presence of the first selectable marker in the host cell, whereby successfully transformed host cells are isolated; and then
d) selecting for the presence of functional second and third selectable markers, whereby a host cell having the correct site-specific chromosomal integration of the gene library is obtained.

In a second aspect the invention relates to polynucleotide constructs for site-specific chromosomal integration of a gene library in a filamentous fungal host cell, said construct comprising an autonomous replication sequence and an integration cassette, said cassette comprising the gene library and a first selectable marker, wherein the cassette is flanked on one side by a non-functional part of a second selectable marker and on the other side by a non-functional part of a third selectable marker.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Nucleic acid construct: The term “nucleic acid construct” or “polynucleotide construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm

(Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position

### BRIEF DESCRIPTION OF DRAWINGS

FIG. 1 shows the plasmid pBAC3155 constructed in Example 1.

FIG. 2 shows the plasmid pBGMH0021 constructed in Example 1.

FIG. 3 shows a photo of an SDS-PAGE; lanes 1-3 show lipase (indicated by the arrow) expressed by a strain having an expression cassette carried on an episomal AMA1-based plasmid (BGMH1000), whereas lanes 4-6 show a significantly thicker lipase band expressed by a strain having an expression cassette integrated into the chromosome of the host strain (BGMH1001; COLS1300) as outlined in Example 2 herein.

### DETAILED DESCRIPTION OF THE INVENTION

The first aspect of the invention relates to methods for site-specific chromosomal integration of a gene library in a filamentous fungal host cell, comprising the steps of:
a) providing a polynucleotide construct comprising an autonomous replication sequence and an integration cassette, said cassette comprising the gene library and a first selectable marker, wherein the cassette is flanked on one side by a non-functional part of a second selectable marker and on the other side by a non-functional part of a third selectable marker;
b) providing a filamentous fungal host cell comprising in its chromosome a non-functional part of the second selectable marker and a non-functional part of the third selectable marker, wherein correct recombinations between the chromosomal non-functional parts of the second and third selectable markers with the respective non-functional parts in the polynucleotide construct will result in functional chromosomal second and third selectable markers;
c) transforming the host cell with the polynucleotide construct and selecting for the presence of the first selectable marker in the host cell, whereby successfully transformed host cells are isolated; and then
   selecting for the presence of functional second and third selectable markers, whereby a host cell having the correct site-specific chromosomal integration of the gene library is obtained.

In a second aspect the invention relates to polynucleotide constructs for site-specific chromosomal integration of a gene library in a filamentous fungal host cell, said construct comprising an autonomous replication sequence and an integration cassette, said cassette comprising the gene library and a first selectable marker, wherein the cassette is flanked on one side by a non-functional part of a second selectable marker and on the other side by a non-functional part of a third selectable marker.

In a preferred embodiment of the invention, the gene library comprises or consists of modified, mutated or variant versions of a gene encoding a parent polypeptide of interest; preferably the parent polypeptide of interest is an enzyme; more preferably it is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

The filamentous fungal host cell of the invention is preferably an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell; more preferably the filamentous fungal host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

It is preferred that the filamentous fungal host cell of the invention has an increased homologous recombination to non-homologous recombination (HR/NHR) ratio; preferably one or more component of the non-homologous end-joining (NHEJ) pathway is repressed or one or more component the homologous recombination (HR) pathway is overexpressed; most preferably an equivalent of the yeast KU70 gene is inactivated.

In a preferred embodiment, the autonomous replication sequence of the invention is the AMA1 sequence from Aspergillus nidulans or a functional derivative thereof.

The selectable markers of the invention are preferably pyrG, niiA and niaD.

### Nucleic Acid Constructs

The present invention also relates to nucleic acid or polynucleotide constructs comprising a gene library of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector comprises an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

### Methods of Production

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### EXAMPLES

Herein we use an autosomally replicating plasmid to transform a large gene library into a filamentous fungus and then to use the double split marker system to ensure site-specific integration of the library into the chromosome of the fungus, thus providing stable and comparable expression yields, even in rich growth medium.

The well-known AMA1 (Autonomous Maintenance) sequence from Aspergillus nidulans makes it possible for a plasmid to replicate episomally in the fungal nucleus. The plasmid we used contained an AMA sequence as well as an integration cassette comprising the gene library and a full-length pyrG gene with a promoter and a terminator, said cassette was flanked on each side by non-functional parts of the niiA and niaD gene, respectively, i.e. the 5″end and promoter of both genes.

The plasmid library was transformed into a pyrG, niiA, niaD minus Aspergillus oryzae host strain (Cols1392) and screened at first in minimal media with urea. This meant that there was only selection for the presence of pyrG, i.e. for successful transformation alone, because there was no need for functional niiA and niaD genes, when the strain used urea as nitrogen source. The tranformed plasmid will probably exist episomally with a tendency to be lost from the nucleus as the fungal cell grows.

When a positive transformant was identified, a large number of spores was plated on to plates with minimal media supplemented with NaNO₃. Only spores, where the integration cassette had successfully recombined into the chromosome of the host cell and reconstituted the niiA and niaD sites in the process, could germinate and survive.

The genomic site-specific integration ensures that the transformants are stable and provides a higher and more uniform expression level, which in turn allows the selection of a single gene of interest from the gene library.

### Example 1

### Construction of an AMA-Based Integration Plasmid

A plasmid that holds both an AMA sequence and regions allowing integration into niiA and niaD in the chromosome of A. oryzase (COls1392) was made as follows.

Insertion of AMA Region into pBGMH14 thus Creating pBAC3155.

The AMA sequence was isolated as a PCR fragment using pEN14286, a derivative of pEN11298 (disclosed in W02008138835) as template and oligos 291012J4 and 291012J5 in a PCR reaction:

291012jvi4:
(SEQ ID NO: 1)
gccgcaattgtggctgcaggtcgaccatgccg
291012jvi5:
(SEQ ID NO: 2)
gccgcaattgaatgataccacagtctagttgac

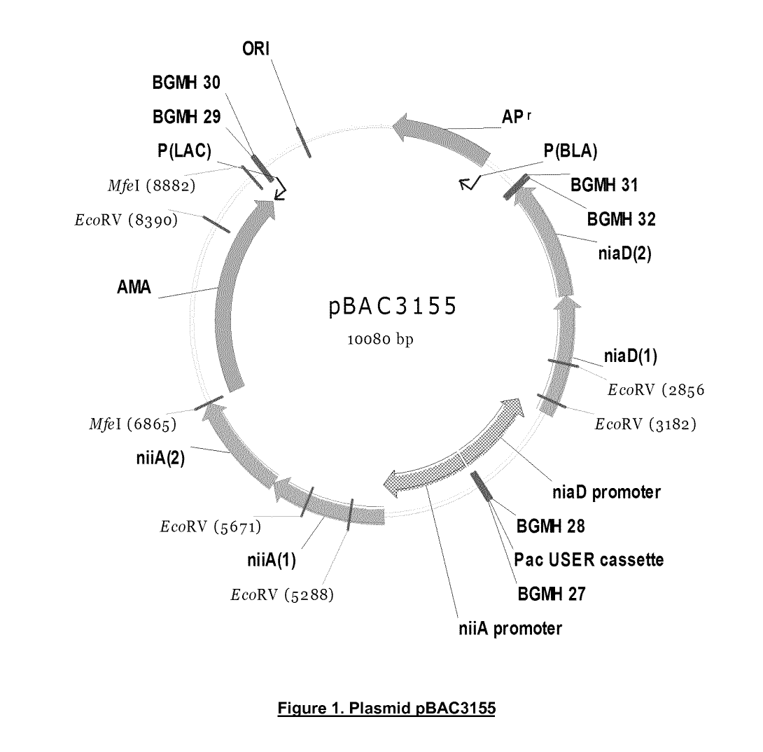

The AMA sequence PCR fragment was cloned using a commercial cloning kit and then transformed into Top10 E. coli cells. A DNA prep was made for the clone and cut with Mfel restriction enzyme. The vector pBGMH14 (WO 2013/119302) was also cut with Mfel and treated with calf intestinal phosphatase. The cut vector and AMA-containing fragment were purified from an agarose gel and ligated overnight. The ligation mixture was transformed into E. coli DH10b, and DNA prep was made of the resulting E. coli clones. The DNA preps were sequenced and cut with restriction enzyme EcoRV to identify a correct clone. The resulting plasmid was named pBAC3155—see FIG. 1.

Insertion of the Lipase Gene into pBAC3155 thus Creating pBGMH0021.

Plasmid pBAC3155 contains a Pacl/Nt.BbvCl Uracil-Specific Excision Reagent or USER™ cassette (Hansen et al., 2011, Appl. Environ. Microbiol. 77(9): 3044-51) which is flanked by part of the A. oryzae niaD gene on one side and part of the A. oryzae niiA gene on the other side (the USER™ trademark is owned by New England Biolabs, USA). Uracil-specific excision enzyme generates a single nucleotide gap at the location of a uracil. The enzyme is a mixture of uracil DNA glycosylase (UDG) and the DNA glycosylase-lyase endonuclease VIII. UDG catalyses the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. The Pacl/Nt.BbvCl USER™ cassette can be linearized with Pacl and Nt.BbvCl; a PCR product with compatible overhangs can then be cloned into this site.

Into the Pacl/Nt.BbvCl USER™ cassette we cloned a fragment containing the NA2tpi promoter from A. oryzae, the T. lanuginosus lipase-encoding gene and a transcriptional terminator. The fragment was PCR amplified from pEN14286 (a derivative of pEN11298) as template using the two uracil-containing primers BGMH155 and BGMH156.

BGMH155:
(SEQ ID NO: 3)
ggacttaauagcgagagagttgaacctggacg
BGMH156:
(SEQ ID NO: 4)
gggtttaaucagatggcccgagaggactattccga

The underlined sequences was used in the USER™ assisted cloning into Pacl/Nt.BbvCl USER™ cassette in pBAC3155.

The amplification reaction was composed of 100 ng of each primer, template DNA (10 ng pEN14286), 1× PfuTurbo® Cₓ Reaction Buffer, 2.5 μl of a blend of dATP, dTTP, dGTP, and dCTP, each at 10 mM, and 2.5 units of PfuTurbo® Cₓ Hot Start DNA Polymerase, in a final volume of 50 μl. The PCR reaction was programmed for 1 cycle at 95° C. for 2 minutes; 40 cycles each at 95° C. for 30 seconds, 55° C. for 30 seconds, and 72° C. for 4 minutes; and a final elongation at 72° C. for 10 minutes.

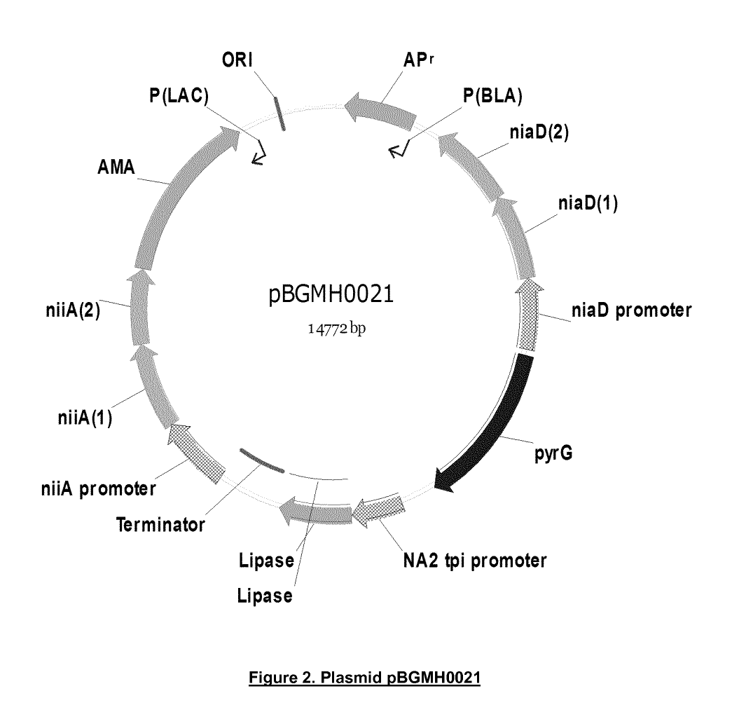

After PCR amplification, 5 μl of 10× NEBuffer 4 and 20 units of Dpn I were added and incubated 1 hour at 37° C. The Dpn I was inactivated at 80° C. for 20 minutes. The PCR product was gel-purified and 50 ng of PCR product, 10 ng of Pacl/Nt.BbvCl digested pBAC3155 and 1 unit of USER™ enzyme in a total volume of 10 μl were incubated for 20 minutes at 37° C. followed by 20 minutes at 25° C. Then 10 μl were transformed into ONE SHOT® TOP10 competent cells. The resulting plasmid was named pBGMH0021—see FIG. 2.

### Example 2

### Transformation of a Filamentous Fungus Strain COLS1392 with pBGMH0021

Plasmid pBGMH0021 was transformed into COLS1392 which was plated on 10 mM urea plates; a transformant was selected, strain BGMH1000, wherein pBGMH0021 exists as an episomal plasmid.

A number of transformants were transferred to new plates with 10 mM urea and after six days spores were harvested by adding 10 ml of water to each plate. 1 ml. spores (amount of spores per ml: approx. 1.5×10⁷) were transferred to plates containing NaNO₃. The plates were incubated at 37° C. for six days providing 10-100 colonies/plate.

Because pyrG, niiA and niaD all need to be functional for a strain to grow on NaNO₃ as the only nitrogen source when no uridine is present in the plates, the colonies that appeared must have repaired both niiA and niaD and have introduced the pyrG gene. For this to be possible, homologous recombination must have taken place in both niiA and niaD, reconstituting both loci in the process. One strain was selected as BGMH1001.

Finally, a pyrG-mutant strain, Cols1300, was selected on uridine containing plates.

| | Selected on | Resulting | niiA; niaD; pyrG | |
|---|---|---|---|---|
| Step # | Plates | Strain | genotype | Result |
| 1 | 10 mM Urea | BGMH1000 | niiA-; niaD- | pBGMH0021 as episomal |
| | | | | plasmid |
| 2 | 10 mM | BGMH1001 | | Repair of niiA and niaD by |
| | NaNO₃ | | | homologous recombination and |
| | | | | chromosomal integration of |
| | | | | pyrG + H. lanuginosa lipase |
| | | | | expression cassette. |
| 3 | 10 mM Urea | Cols1300 | niiA-; niaD-; | Selection of pyrG-mutant. |
| | 10 mM | | pyrG- | |
| | Uridine | | | |

### Example 3

### Comparison of Lipase Yields from Episomal vs. Integrated Expression Cassette

The strains constructed in Example 2 were grown in MTP in 200 μl YPM-media (+Urea) for 3 days at 34° C. The expression level of the H. lanuginosa lipase from an episomal AMA1-based plasmid-borne expression cassette (strain BGMH1000) and from a chromosomally site-specifically integrated expression cassette (strain BGMH1001/COLS1300) were compared by SDS-PAGE.

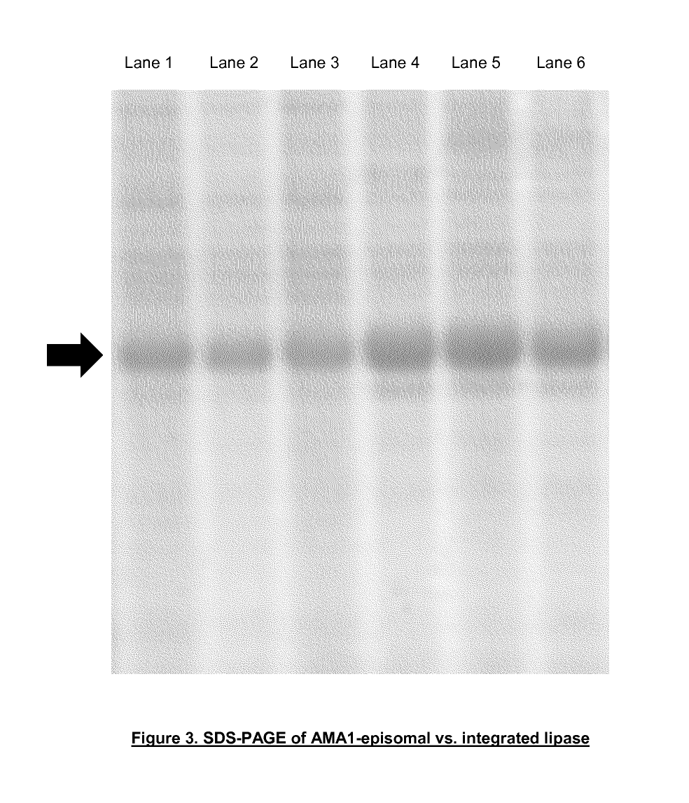

A photo of the SDS-PAGE gel is shown in FIG. 3. The lipase-band is indicated by the arrow; lanes 1-3 show the episomal AMA1-based plasmid-expression, whereas lanes 4-6 show a surprisingly thicker lipase band expressed by the chromosomally integrated cassette according to the invention.

1. A method for site-specific chromosomal integration of a gene library in a filamentous fungal host cell, comprising the steps of:
(a) providing a polynucleotide construct comprising an autonomous replication sequence and an integration cassette, said cassette comprising the gene library and a first selectable marker, wherein the cassette is flanked on one side by a non-functional part of a second selectable marker and on the other side by a non-functional part of a third selectable marker;
(b) providing a filamentous fungal host cell comprising in its chromosome a non-functional part of the second selectable marker and a non-functional part of the third selectable marker, wherein correct recombinations between the chromosomal non-functional parts of the second and third selectable markers with the respective non-functional parts in the polynucleotide construct will result in functional chromosomal second and third selectable markers;
(c) transforming the host cell with the polynucleotide construct and selecting for the presence of the first selectable marker in the host cell, whereby successfully transformed host cells are isolated; and then
(d) selecting for the presence of functional second and third selectable markers, whereby a host cell having the correct site-specific chromosomal integration of the gene library is obtained.

2. The method of claim 1, wherein the gene library comprises or consists of modified, mutated or variant versions of a gene encoding a parent polypeptide of interest;
preferably the parent polypeptide of interest is an enzyme; more preferably it is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

3. The method of claim 1, wherein the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phiebia, Piromyces, Pleurotus, Schizophyllum, Talaromyces, Thermoascus, Thielavia, Tolypocladium, Trametes, or Trichoderma cell.

4. The method of claim 3, wherein the filamentous fungal host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phiebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

5. The method of claim 1, wherein the filamentous fungal host cell has an increased homologous recombination to non-homologous recombination (HR/NHR) ratio; preferably one or more component of the non-homologous end-joining (NHEJ) pathway is repressed or one or more component the homologous recombination (HR) pathway is overexpressed; most preferably an equivalent of the yeast KU70 gene is inactivated.

6. The method of claim 1, wherein the autonomous replication sequence is the AMA1 sequence from Aspergillus nidulans or a functional derivative thereof.

7. The method of claim 1, wherein the selectable markers are pyrG, niiA and niaD.

8. A polynucleotide construct for site-specific chromosomal integration of a gene library in a filamentous fungal host cell, said construct comprising an autonomous replication sequence and an integration cassette, said cassette comprising the gene library and a first selectable marker, wherein the cassette is flanked on one side by a non-functional part of a second selectable marker and on the other side by a non-functional part of a third selectable marker.

9. The polynucleotide construct of claim 8, wherein the gene library comprises or consists of modified, mutated or variant versions of a gene encoding a parent polypeptide of interest; preferably the parent polypeptide of interest is an enzyme; more preferably it is an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

10. The polynucleotide construct of claim 8, wherein the autonomous replication sequence is the AMA1 sequence from Aspergillus nidulans or a functional derivative thereof.

11. The polynucleotide construct of claim 8, wherein the selectable markers are pyrG, niiA and niaD.
